(19) United States
(12) Patent Application Publication
(10) Pub. No.: US 2003/0100499 A1
(43) Pub. Date: May 29, 2003

(54) In vitro model for the treatment of restenosis

(76) Inventor: Stephen E. Epstein, Rockville, MD (US)

Correspondence Address: HELLER EHRMAN WHITE & MCAULIFFE LLP, SUITE 300, 101 ORCHARD RIDGE DR., GAITHERSBURG, MD, 20878-1917, US

(21) Appl. No.: 10/230,390

(22) Filed: Aug. 29, 2002

Related U.S. Application Data
(60) Provisional application No. 60/315,725, filed on Aug. 30, 2001.

(51) Int. Cl. A61K 48/00; A61K 38/17; C12Q 1/00; C12M 1/34

(52) U.S. Cl. 514/12; 514/44; 435/4; 435/287.1

(57) ABSTRACT

The invention provides in vitro methods useful for screening compositions and methodologies in the treatment and prevention of coronary artery disease and, in particular, restenosis. The invention also provides devices and compositions that are used in the methods.

### FIELD OF THE INVENTION

The invention provides in vitro methods useful for screening compositions and methodologies in the treatment and prevention of coronary artery disease and, in particular, restenosis. The invention also provides devices and compositions that are used in the methods.

### BACKGROUND OF THE INVENTION

Coronary artery disease is a disease that is endemic in Western society. In this disease the arteries that supply blood to the heart muscle become narrowed by deposits of fatty, fibrotic, or calcified material on the inside of the artery. The build up of these deposits is called atherosclerosis. Atherosclerosis reduces the blood flow to the heart, starving the heart muscle of oxygen and leading to angina pectoris (chest pain), myocardial infarction (heart attack), and/or congestive heart failure. One common treatment to clear arteries blocked by atherosclerosis is balloon angioplasty, more formally referred to as percutaneous transluminal coronary angioplasty (PTCA). This treatment involves opening up a blocked artery by inserting and inflating a small balloon, which compresses and rearranges the blocking plaque against the arterial wall. After deflation and removal of the balloon, the arterial lumen is enlarged, thereby improving blood flow. About one million angioplasty procedures are performed each year.

In a significant number of angioplasty patients the treated artery narrows again within six months of the procedure in a process called restenosis. Restenosis begins soon after angioplasty, wherein the increased size of the vascular lumen (the open channel inside the artery) becomes gradually occluded by the proliferation of smooth muscle cells. Approximately 20 to 30% of angioplasty patients experience restenosis to the extent that they must undergo repeated angioplasty or even coronary bypass surgery.

Restenosis has a complex pathology, triggered by the stretch-induced injury of the vessel walls during balloon inflation, which stimulates smooth muscle cell migration and proliferation, and thereby leads to neointimal accumulation (which constitutes the restenotic lesion). Additional processes contributing to restenosis include inflammation and accumulation of extracellular matrix. Remodeling of the vessel wall, leading to narrowing of the vessel, is a critically important component of restenosis. However, this is totally eliminated by the implacement of a stent at the site of angioplasty, which prevents the vessel from remodeling. Stenting has become almost routine, being performed in many centers in over 70% of all angioplasty procedures.

Currently, new methods for treatment of restenosis are developed using an in vivo model such as the pig or rat. These methods are slow, laborious and expensive because of the time it takes to breed appropriate animals, the time required to promote a restenotic state, the complexity of the surgical procedures necessary to develop the model, and the time needed to determine the effects of an intervention. Consequently, the rate of developing new methods for the treatment or prevention of restenosis is severely limited. It is apparent, therefore, that a new model is needed whereby the effects of therapeutic agents or drugs can be tested quickly and economically in order to determine whether a full-scale animal trial is warranted.

### SUMMARY OF THE INVENTION

It is therefore an object of this invention to provide improved methods and apparatus for the screening and identification of therapeutic agents in the treatment and prevention of coronary artery disease.

It is also an object of this invention to provide an in vitro model for the identification of agents beneficial in treating restenosis. In accordance with this goal the inventors have developed an in vitro model simulating the arterial vessel wall, that allows testing of the efficacy of a broad array of agents (for example, small molecules, proteins, naked DNA, transgenes carried in viral vectors, and cellular strategies) on inhibiting the proliferation and migration of smooth muscle cells or endothelial cells cultured on this surrogate vessel.

In one embodiment, the invention provides a method for determining the potential of a therapeutic agent for treating coronary artery disease, comprising (i) incubating cells in a well containing culture medium, where the well is divided into an upper chamber and a lower chamber by a porous membrane and where the cells are initially present in the upper chamber, where the well contains the therapeutic agent and where the lower chamber contains a chemoattractant agent; (ii) determining the number of cells that migrate into the lower chamber after incubation; and (iii) comparing the rate of proliferation of those cells that migrate and those cells that do not migrate; whereby the comparison indicates the potential of therapeutic agent for the treatment or prevention of coronary artery disease. The porous membrane may contain a biocompatible coating suitable for placement in a blood vessel, and the coating may be impregnated with the therapeutic agent. The coating may comprise, for example, a collagen or a hydrogel.

In another embodiment the cells in the well are selected from the group consisting of smooth muscle cells, endothelial cells, mesenchymal cells, monocytes, macrophages, and T cells and combinations of these cells.

In still another embodiment, the therapeutic agent comprises at least one composition selected from the group consisting of cells, nucleic acids, antibodies, proteins, peptide fragments, viral vectors, drugs, and chemical substances. In a further embodiment, the therapeutic agent comprises cells comprising a gene for the treatment or prevention of coronary artery disease.

In yet another embodiment, the cells comprise at least one cell type selected from the group consisting of mesenchymal cells, endothelial progenitor cells and stem cells.

In still another embodiment, the coronary artery disease is atherosclerosis, stenosis or restenosis.

In a further embodiment, at least one surface of said membrane is contacted by a layer of stent material. The stent material may further comprise a biocompatible coating suitable for placement in a blood vessel.

In a still further embodiment the pores in the porous membrane are about 5 μm in diameter.

In yet another embodiment the chemoattractant comprises at least one growth factor selected from the group consisting of PDGF-BB, IGF-1, EGF, FGF, HGF, NGF, TGF and VEGF.

In a further embodiment, there is provided a device for evaluating a therapeutic agent for treatment or prevention of coronary artery disease comprising a well suitable for culturing cells with culture media disposed within the well, where the well comprises an upper chamber and a lower chamber separated by a porous membrane, where the porous membrane is coated with a biocompatible coating suitable for placement in a coronary blood vessel, a chemoattractant agent disposed in the lower chamber, and cells disposed within the upper chamber. At least one surface of the membrane may optionally be contacted by a layer of stent material. In a further embodiment the well contains a putative therapeutic agent for the treatment, prevention, or amelioration of coronary artery disease.

Other objects, features and advantages of the present invention will become apparent from the following detailed description. It should be understood, however, that the detailed description, while indicating preferred embodiments of the invention, is given by way of illustration only, since various changes and modifications within the spirit and scope of the invention will become apparent to those skilled in the art from this detailed description.

### BRIEF DESCRIPTION OF THE DRAWINGS

FIG. 1 shows a diagrammatic representation of a coronary artery that has been stented, with the stent having a coating on it. The stent with its coating abuts the smooth muscle cells of the arterial media.

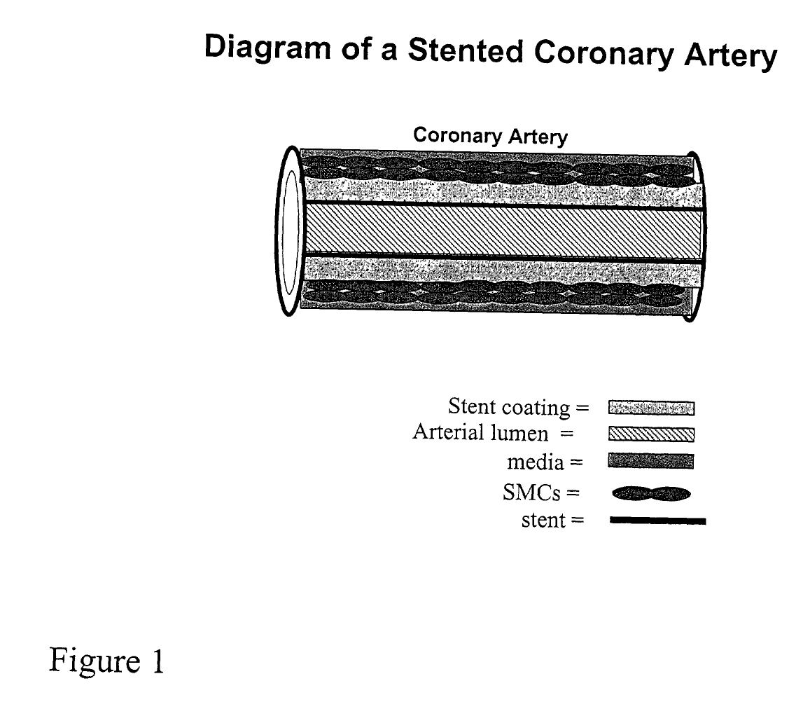

FIG. 2 shows a diagrammatic representation of a coronary artery that has been stented, with the stent having a coating on it. The stent with its coating abuts the smooth muscle cells of the arterial media. In this example plasmid DNA or adenoviral vector is impregnated into the stent coating.

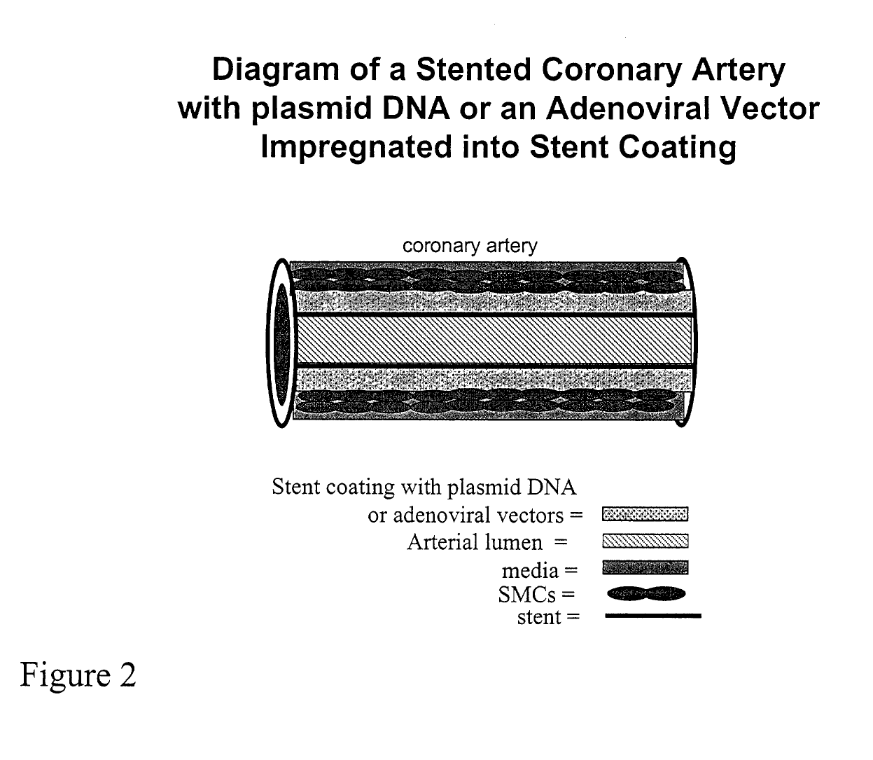

FIG. 3 shows a diagrammatic representation of a coronary artery that has been stented, with the stent having a coating on it. The stent with its coating abuts the smooth muscle cells of the arterial media. In this example endothelial progenitor cells (EPCs) or stem cells are impregnated into the stent coating.

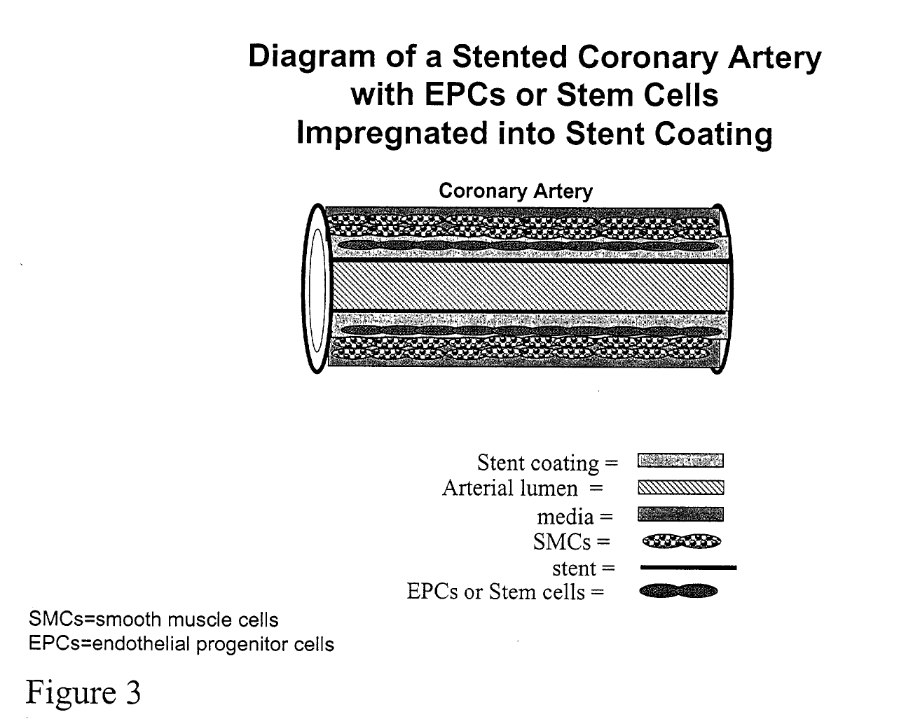

FIG. 4 shows a diagrammatic representation of an in vitro model of a coronary artery that has been stented, with the stent having a coating on it. The stent with its coating abuts the smooth muscle cells that simulate the arterial media. The membrane on which the stent sits separates the upper chamber of the well from the lower chamber. The lower chamber simulates the arterial lumen. The well is filled with culture media while PDGF-BB is added to the media in the lower chamber.

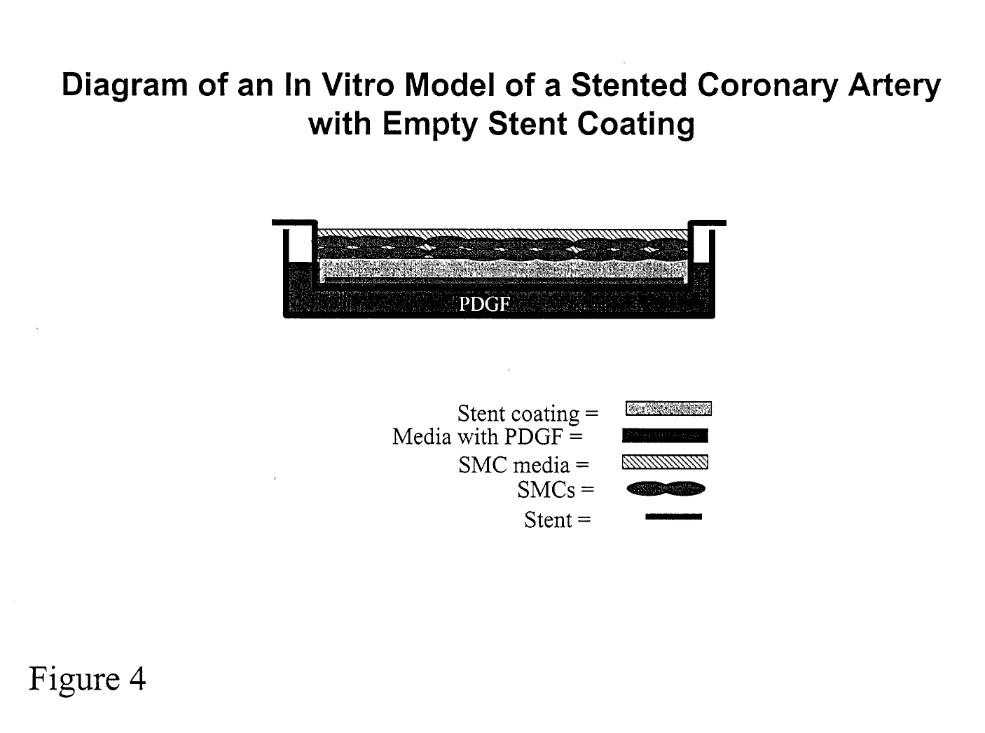

FIG. 5 shows a diagrammatic representation of an in vitro model of a coronary artery that has been stented, with the stent having a coating on it. The stent with its coating abuts the smooth muscle cells that simulate the arterial media. The membrane on which the stent sits separates the upper chamber of the well from the lower chamber. The lower chamber simulates the arterial lumen. In this example, plasmid DNA or adenoviral vectors are impregnated into the stent coating..

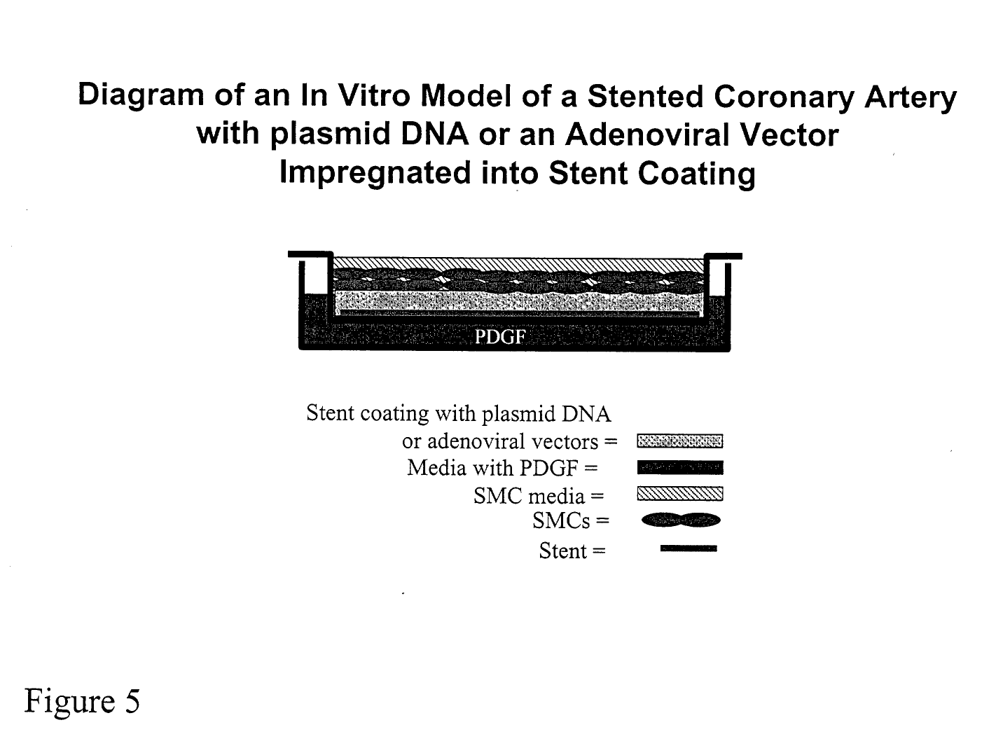

FIG. 6 shows a diagrammatic representation of an in vitro model of a coronary artery that has been stented, with the stent having a coating on it. The stent with its coating abuts the smooth muscle cells that simulate the arterial media. The membrane on which the stent sits separates the upper chamber of the well from the lower chamber. The lower chamber simulates the arterial lumen. In this example, endothelial progenitor cells (EPCs) or stem cells are impregnated into the coating the covering stent.

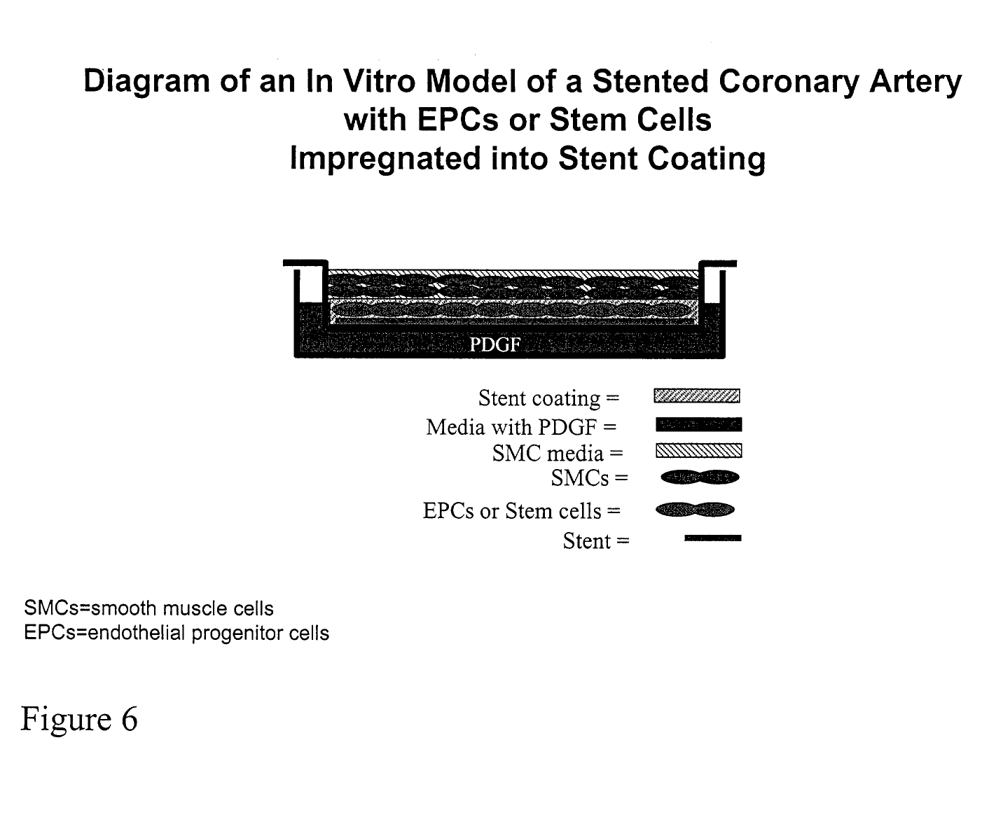

### DETAILED DESCRIPTION OF THE INVENTION

The invention provides in vitro models for identifying and testing therapeutic agents for use in the treatment and prevention of coronary artery disease and, specifically, restenosis.

In Vitro Model for Assessing Efficacy of Putative Treatments for Restenosis

The inventors have found that methods for treating restenosis may first be assayed using an in vitro model to test the efficacy of putative therapeutic agents before resorting to in vivo trials on animal models. The model system utilizes cell culture strategy. A well suitable for culturing cells is divided into upper and lower chambers by a porous membrane. The upper chamber mimics the environment of the arterial wall while the lower chamber, inoculated with a chemoattractant, mimics the environment of the arterial lumen. Smooth muscle cells are cultured on the membrane, and this arrangement mimics the artery. The well is filled with media suitable for culturing the cells. Suitable media are well known in the art and are commercially available from, for example, Invitrogen (Carlsbad, Calif.), Fisher Scientific, Sigma (St. Louis, Mo.) and Hyclone (Logan, Utah). An example of a suitable culture medium is RPMI-1640 supplemented with 10% fetal bovine serum (FBS). One skilled in the art will be aware that other media also are suitable for use in the well. Using this design, the effect of any putative strategy on the migration of smooth muscle cells into the arterial lumen and the development of restenosis can be studied.

The membrane separating the upper and lower chambers contains pores approximately 5 μm in diameter, although other pore sizes may also be used. The membrane provides a platform for administrating the therapeutic agent tested. The membrane may be essentially any suitable material that is compatible with culture of cells. Suitable membrane materials include, but are not limited to, polycarbonate, cellulose and polyvinylidene fluoride (PVDF). In each instance the membrane allows the free diffusion of culture media, cytokines and other cellular factors across the membrane.

The membrane may be coated with a compound used to cover stents. This compound may serve as a therapy or preventive for restenosis, or as a vehicle into which a therapeutic agent can be impregnated. Suitable coatings are well known in the art. For example, the coating may be collagen or may be a hydrogel. The model system also may contain stent material that contacts the membrane to mimic the effect of a stented artery. The stent may, for instance, be cut to the contours of the membrane, flattened and laid on top of the membrane. The stent may be covered by the coating, in addition to or in place of the membrane coating. The stent mimics the stent in a vessel allowing a greater approximation of the effects of the treatment in vivo.

The coating can be impregnated with a compound or agent to be tested as a therapy for atherosclerosis, stenosis and/or restenosis. The compound or agent may be cells such as endothelial progenitor cells, stem cells, or mesenchymal cells which can express genes that may have potential as treatments for atherosclerosis, stenosis and/or restenosis. These genes typically are transgenes that are inserted into the cell by, for example, transfection, but also may be endogenous genes that are highly expressed or exhibit enhanced expression due to the presence, for example, of exogenous regulatory elements (so-called gene activation technology). The compound may be “naked” DNA that encodes a therapeutic peptide, polypeptide or protein. The compound may also be a protein, polypeptide, peptide or small molecule therapeutic. The compound also may be a viral vector carrying a transgene or a drug.

By culturing appropriate cells on the membrane it is possible to test the therapeutic properties of the compound embedded in the coating. The cells may be, for example, smooth muscle cells, endothelial cells, or a mixture of endothelial cells and smooth muscle cells. The cells may also be a mixture of endothelial cells, smooth muscle cells, and one or more types of inflammatory cells such as activiated monocytes/macrophages or T cells. The cells also may be any other cells that provide a means to study the anti-migrational, anti-proliferative, and/or anti-inflammatory effects of the agent tested. In this manner cells that migrate through the pores in the membrane (or membrane and stent) will absorb the agent impregnated in the coating and affect the proliferation of those cells upon migration to the lower chamber, which effectively form a neointima.

To study the effects on migration a chemoattractant agent is placed in the lower chamber. Suitable chemoattractants are well known in the art. The chemoattractant may be platelet derived growth factor (PDGF), insulin-like growth factor (IGF), or another growth factor such as epidermal growth factor (EGF), fibroblast growth factor (FGF), hepatocyte growth factor (HGF), nerve growth factor (NGF), transforming growth factor (TGF), vascular endothelial growth factor (VEGF) or any other agent inducing cell growth and migration. Combinations of chemoattractants also may be used.

This design allows for a quantification of the therapeutic value of the therapeutic agent by allowing measurements of cells migrating into the lower chamber at 6, 8 or 12 hours (or other appropriate time points) after seeding of the cells. This design also allows a measure of the effects of the therapeutic agent on the proliferation of the cells in the upper chamber (simulating the effect of the therapeutic agent on smooth muscle cells located in the vessel media) by allowing the quantification of those cells at day 1, 3, 7 or 14 (or other appropriate time points). Further, this design also provides a measure of the effects of the therapeutic agent on the proliferation of the cells that have migrated into the lower chamber (simulating the effect of the therapeutic agent on smooth muscle cells located in the growing neointima that becomes the restenotic lesion) by allowing the quantification of those cells at day 1, 3, 7 or 14 (or other appropriate time points). Cells may be counted by known methods, including cell counting after trypsinization or by tritiated thymidine incorporation assay.

By simulating the environment of a damaged arterial vessel and by placing a chemoattractant in the lower chamber, the therapeutic effect of the agent on the membrane or stent in (i) inhibiting proliferation of cells located in the vessel wall, (ii) inhibiting migration of cells into the vessel lumen, and (iii) inhibiting proliferation of cells once gaining access to the vessel lumen, can be estimated. Once an agent has been shown to inhibit the proliferation of cells located in the upper chamber, inhibit the migration of cells into the lower chamber, and/or inhibit the proliferation of cells once gaining access into the lower chamber, the value of the agent therapeutically may be further tested by its efficacy in established in vivo models such as the pig or rat.

As may be appreciated the ability of other researchers to use the model described herein will greatly advance the research on therapeutic agents to treat restenosis. To this end it is contemplated that kits or devices meeting the description of the invention are also claimed. Such kits or devices contain appropriate culture vessels and membranes or filters of appropriate size to separate the well into upper and lower chambers. In addition, the kits or devices may provide cells that may be cultured and seeded onto the membrane or stent. In the kit, these cells may be provided in a frozen form suitable for long-term storage. In addition, the kits or devices provide stents that fit the membrane comprising the bottom of the membrane. In addition, it is contemplated that appropriate media will be available such that the researcher using such kit or device can easily maintain the model system allowing for optimal cell growth. In addition, appropriate coating materials may be provided such that the researcher, using such kit or device with whatever coating is desired, can easily test the efficacy of agents embedded in the coating. Such kits or devices will allow the easy quantification of the effects of any therapeutic agent by applying the agent to the membrane, or to the coating, or to the coating of the stent, and measuring the proliferation and migration of cells at the appropriate time points.

The invention disclosed herein provides an in vitro model to study the effects of various therapeutic agents including cells (such as progenitor endothelial cells or stem cells) genetically engineered cells that express potentially therapeutic agents, proteins, peptides, small molecules, viral agents, naked DNA, drugs, or any other compound. The in vitro model will provide an economical way to test the effects of therapeutic agents to be used in treating atherosclerotic disease. The effects of the agents on neointima formation can be studied in order to determine whether full scale, in vivo, trials in animal models should be pursued.

What is claimed is:

1. A method for determining the potential of a therapeutic agent for treating coronary artery disease, comprising:
incubating cells in a well containing culture medium wherein said well is divided into an upper chamber and a lower chamber by a porous membrane and wherein said cells are initially present in said upper chamber;
wherein said well contains the therapeutic agent;
wherein said lower chamber contains a chemoattractant agent;
determining the number of cells that migrate into said lower chamber after incubation; and
comparing the rate of proliferation of those cells that migrate and those cells that do not migrate;
whereby said comparison indicates the potential of said therapeutic agent for the treatment or prevention of coronary artery disease.

2. The method of claim 1 wherein the porous membrane contains a biocompatible coating suitable for placement in a blood vessel.

3. The method of claim 2 wherein said coating is impregnated with said therapeutic agent.

4. The method of claim 2 or claim 3 wherein said coating comprises a collagen or a hydrogel.

5. The method of claim 1 wherein said therapeutic agent comprises at least one composition selected from the group consisting of cells, nucleic acids, antibodies, proteins, peptide fragments, viral vectors, drugs, chemical substances.

6. The method of claim 1 wherein the therapeutic agent comprises cells comprising a gene for the treatment, prevention, or amelioration of coronary artery disease.

7. The method of claim 6 wherein said cells comprise at least one cell type selected from the group consisting of mesenchymal cells, endothelial progenitor cells and stem cells.

8. The method of claim 1 wherein the coronary artery disease is atherosclerosis, stenosis or restenosis.

9. The method of claim 1 wherein at least one surface of said membrane is contacted by a layer of stent material.

10. The method of claim 9 wherein said stent material comprises a biocompatible coating suitable for placement in a blood vessel.

11. The method of claim 1 wherein the pores in said porous membrane are about 5 μm in diameter.

12. The method of claim 1 wherein said cells are selected from the group consisting of smooth muscle cells, endothelial cells, mesenchymal cells, monocytes, macrophages, and T cells and combinations thereof.

13. The method of claim 1 wherein the chemoattractant comprises at least one growth factor selected from the group consisting of PDGF-BB, IGF-1, EGF, FGF, HGF, NGF, TGF and VEGF.

14. A device for evaluating a therapeutic agent for treatment of coronary artery disease comprising:
a well suitable for culturing cells, wherein said well comprises an upper chamber and a lower chamber separated by a porous membrane, wherein said porous membrane is coated with a biocompatible coating suitable for placement in a coronary blood vessel;
culture media disposed within said well;
a chemoattractant agent disposed in said lower chamber, and
cells disposed within said upper chamber.

15. The device of claim 14 wherein at least one surface of said membrane is contacted by a layer of stent material.

16. The device of claim 14 wherein said well contains a putative therapeutic agent for the treatment of coronary artery disease.
